(19) United States
(12) Patent Application Publication
(10) Pub. No.: US 2016/0199087 A1
(43) Pub. Date: Jul. 14, 2016

(54) ADJUSTABLE TISSUE CUTTER TOOL

(71) Applicants: PETER SCOTT BORDEN, Torrance, CA (US); Matthew Tarnay, Torrance, CA (US)

(72) Inventors: PETER SCOTT BORDEN, Torrance, CA (US); Matthew Tarnay, Torrance, CA (US)

(21) Appl. No.: 14/406,753

(22) PCT Filed: Jun. 20, 2013
(86) PCT No.: PCT/US13/46886

§ 371 Date: Dec. 10, 2014

Related U.S. Application Data
(60) Provisional application No. 61/662,324, filed on Jun. 20, 2012.
(60) Provisional application No. 61/749,833, filed on Jan. 7, 2013.

(51) Int. Cl. A61B 17/32

(52) U.S. Cl. CPC A61B 17/320016; A61B 2017/00371

(57) ABSTRACT

An adjustable, tissue cutting tool for use in arthroscopic surgery and including a distal-end cutting mechanism adapted to be adjustably angled relative to the principal axis of the tool in order to reach tight, difficult to reach areas of tissue damage within a joint and to thereby minimize risk of iatrogenic, articular cartilage damage.

### FIELD OF INVENTION

The invention relates generally to a device for use in arthroscopic surgical removal of soft tissue damage within a joint, e.g., a knee of a human or animal.

### BACKGROUND OF INVENTION

Arthroscopy is a medical term used to describe a minimally-invasive procedure in which a camera is inserted through a narrow incision into a body joint for the purpose of accurate diagnosis and treatment of various intra-articular injuries. The camera is then used to guide the use of an instrument also inserted into the joint through a narrow incision. There currently are various instruments designed for arthroscopic removal of damaged tissue available. These devices are rigid, reusable instruments and can be acquired with the cutting end fixed at various angles. The surgeon selects a particular instrument set at a particular angle based on the particular cutting task being performed at that moment.

Surgical devices providing some degree of adaptability in the form of flexibility in the shaft of the device are known. For example, U.S. Pat. No. 3,915,169 (“the '169 patent”) describes a knife specifically designed for removing meniscus from knee joints that has a “malleable” shank. U.S. Pat. No. 6,139,563 (“the '563 patent”) describes a forceps-like device for grasping, securing and occluding body tissues and conduits which features a shaft that can be bent and adjusted to minimize its intrusion and allow for better positioning of the jaws of the device within the body.

### SUMMARY OF INVENTION

Embodiments of an adjustable arthroscopic tissue-cutting device that allows for angulation of the cutting head of the device at the time of surgery by the using surgeon (or surgical staff) to the degree desired by the surgeon described herein provide the first known such device.

The Adjustable Tissue Cutter Tool can be used in various joints such as the knee, shoulder, elbow, wrist, and ankle.

Certain embodiments of the device feature a knob-controlled mechanism to adjust the angle of the cutting end relative to the shaft of the instrument. Other embodiments feature a shaft that has a rigid/flexible region, which is manipulated into a desired angle by the using surgeon (or surgical staff). Such embodiments of the device allow for a potentially-disposable single-use device that avoids the common problem of diminishing sharpness of the cutting edge associated with reusable devices. In some embodiments, the shaft of the device preferably tapers towards the end for the advantage of reaching tight, difficult areas in and near the joint to remove damaged tissue with less potential for iatrogenic, articular cartilage damage.

These and other embodiments, features, aspects, and advantages of the invention will become better understood with regard to the following description, appended claims and accompanying drawings.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a side view of a preferred embodiment adjustable tissue cutter with a rigid/flexible shaft region with the shaft in its initial, straight configuration;

FIG. 2 is a top view of the FIG. 1 embodiment with the rigid/flexible shaft region bent to provide a 30° horizontal offset of the distal end from the centerline of the instrument;

FIG. 3 is a cut-away, top view of the rigid/flexible portion of the shaft and adjoining regions of the FIG. 1 embodiment configured as in FIG. 2;

FIG. 4 is a cut-away, close-up, side view of the distal end of the FIG. 1 embodiment oriented at a downward angle;

FIG. 5 is a close-up, side view of the distal end of the FIG. 1 embodiment;

FIG. 6 is a side view of a preferred embodiment adjustable tissue cutter with gear-controlled adjustment of the horizontal angle of the distal-end cutter with that distal-end cutter in its initial, straight configuration;

FIG. 7 is a top view of the FIG. 6 embodiment with the distal-end cutter in its initial, straight configuration;

FIG. 8 is a perspective view of the FIG. 6 embodiment with the distal-end cutter in its initial, straight configuration;

FIG. 9 is three close-up, top views of the distal end of the FIG. 6 embodiment with the distal-end cutter (from top to bottom) (A) offset to the left from the centerline of the instrument; (B) in its initial, straight configuration, corresponding to FIG. 7; and (C) offset to the right from the centerline of the instrument;

FIG. 10 is a cut-away, close-up, perspective view of the actuator end of the FIG. 6 embodiment.

FIG. 11 is a cut-away, close-up, perspective view of the distal end of the FIG. 6 embodiment.

FIG. 12 is a second cut-away, close-up, perspective view of the distal end of the FIG. 6 embodiment.

FIG. 13 is an exploded, cut-away, close-up, perspective view of the distal end of the FIG. 6 embodiment.

FIG. 14 is a cut-away, close-up, perspective view of the control-knob portion of the actuator end of the FIG. 6 embodiment with the control knob in an active (unlocked or disengaged) position.

FIG. 15 is a cut-away, close-up, perspective view of the control-knob portion of the actuator end of the FIG. 6 embodiment with the control knob in its initial, resting (locked or engaged) position.

FIG. 16 is a cut-away, close-up, perspective view of the central adjustment-transfer portion of the FIG. 6 embodiment.

FIG. 17 is a side view of a preferred embodiment adjustable tissue cutter with wire-controlled adjustment of the horizontal and vertical angles of the distal-end cutter with that distal-end cutter tipped upward relative to the centerline of the device;

FIG. 18 is a perspective view of the FIG. 17 embodiment with the distal-end cutter in its initial, straight configuration;

FIG. 19 is a cut-away, close-up, perspective view of the actuator end of the FIG. 17 embodiment.

FIG. 20 is a cut-away, close-up, perspective view of the distal end of the FIG. 17 embodiment.

FIG. 21 is an exploded, close-up, perspective view of the distal end of the FIG. 17 embodiment.

Reference symbols or names are used in the figures to indicate certain components, aspects or features shown therein. Reference symbols common to more than one Figure indicate like components, aspects or features shown therein.

### DETAILED DESCRIPTION

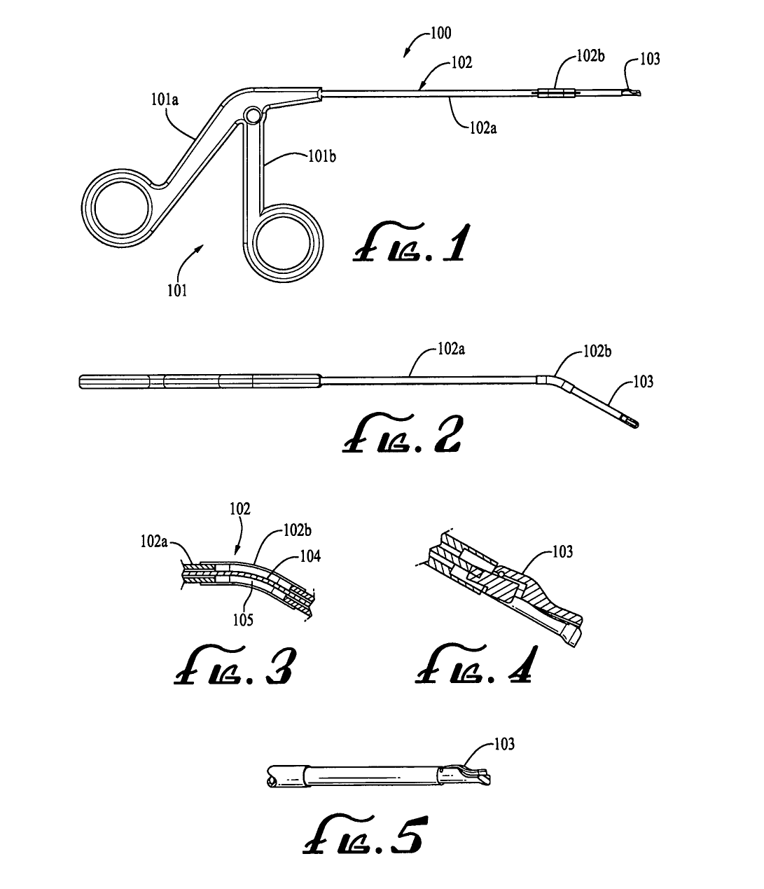

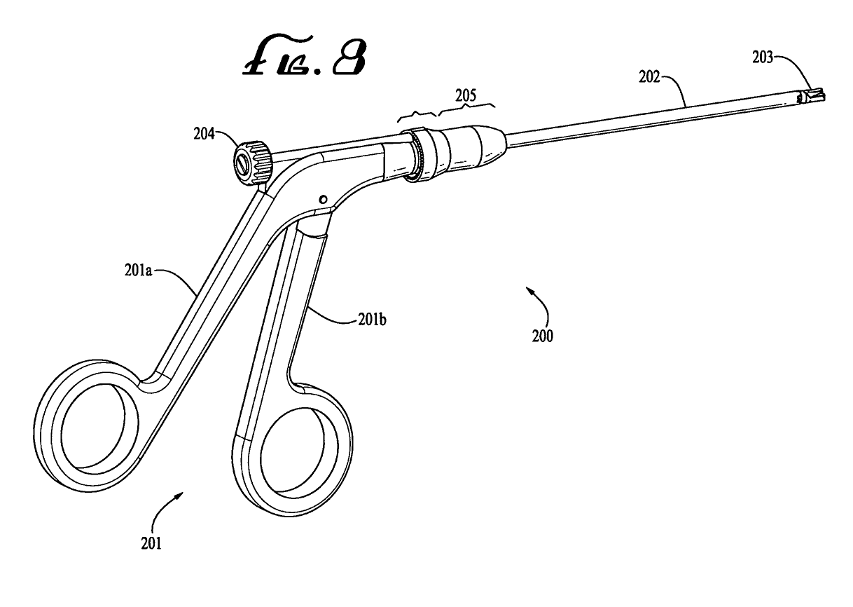

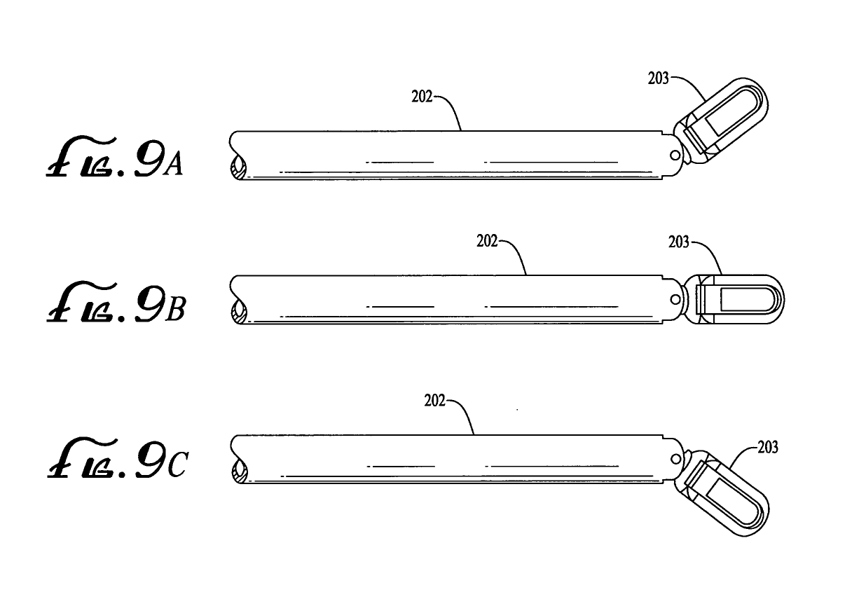

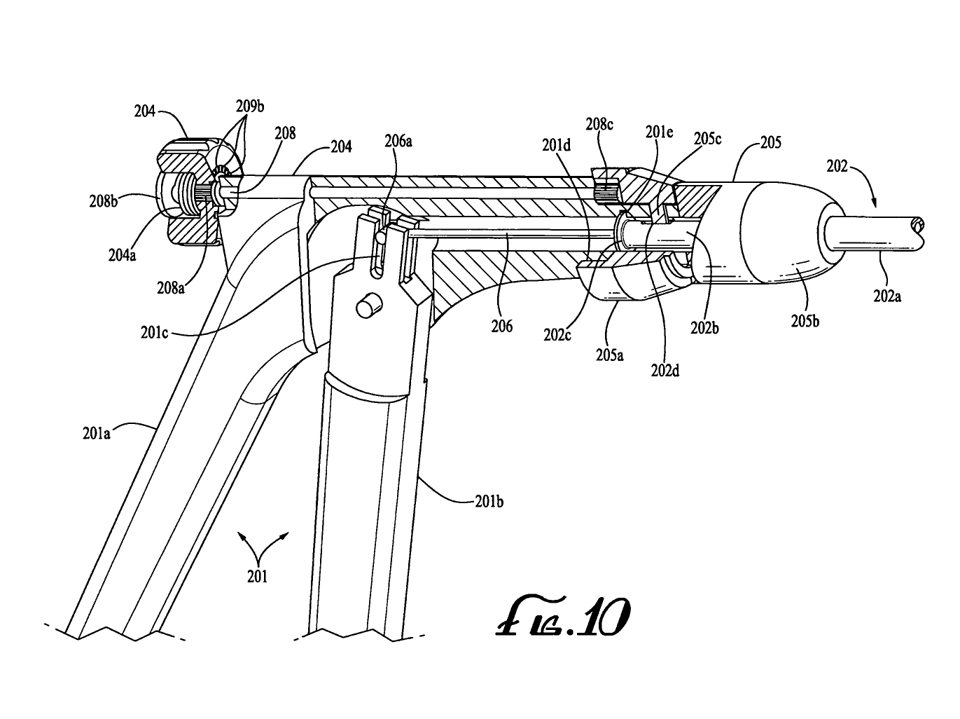

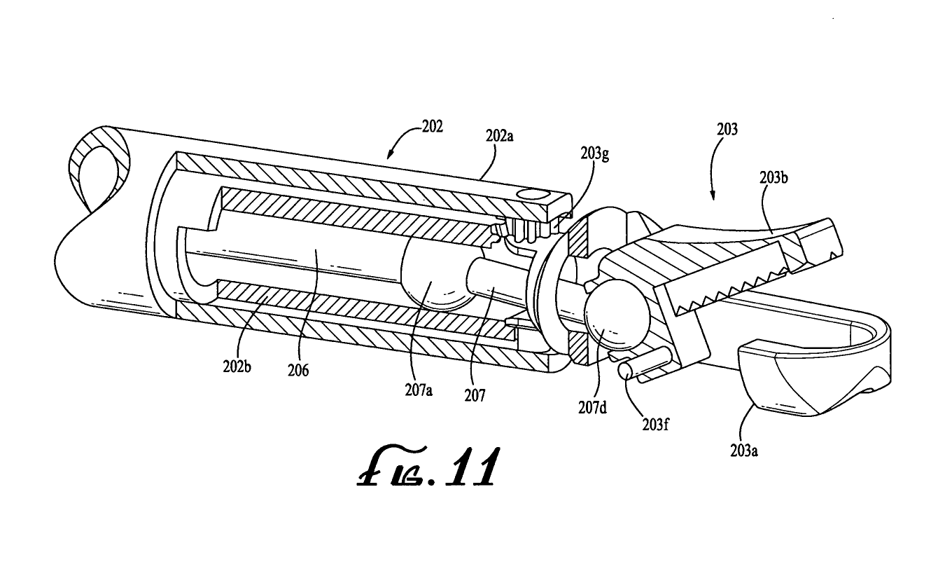

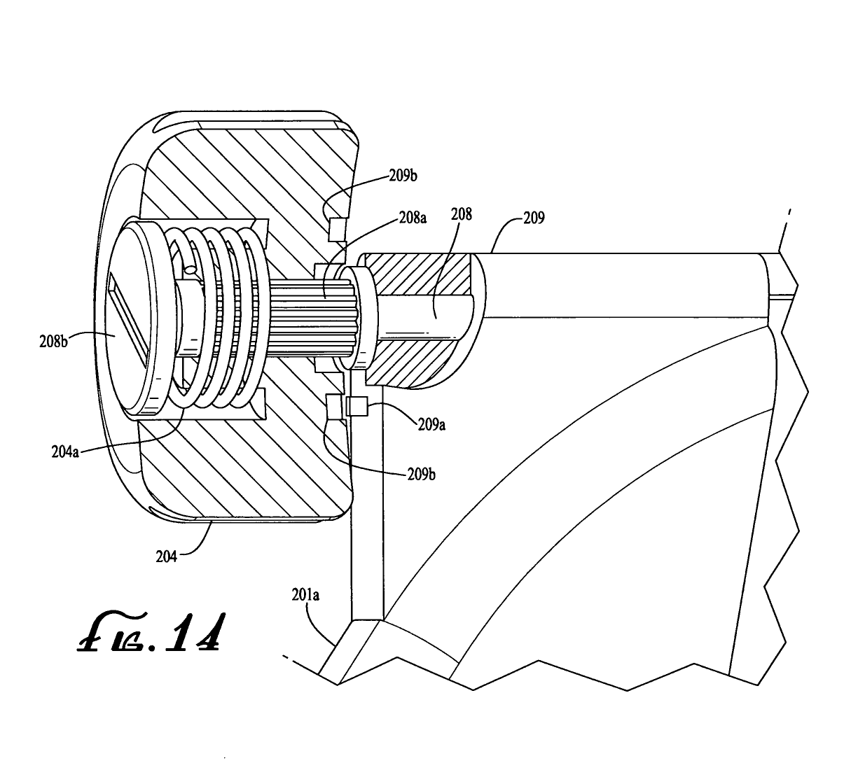

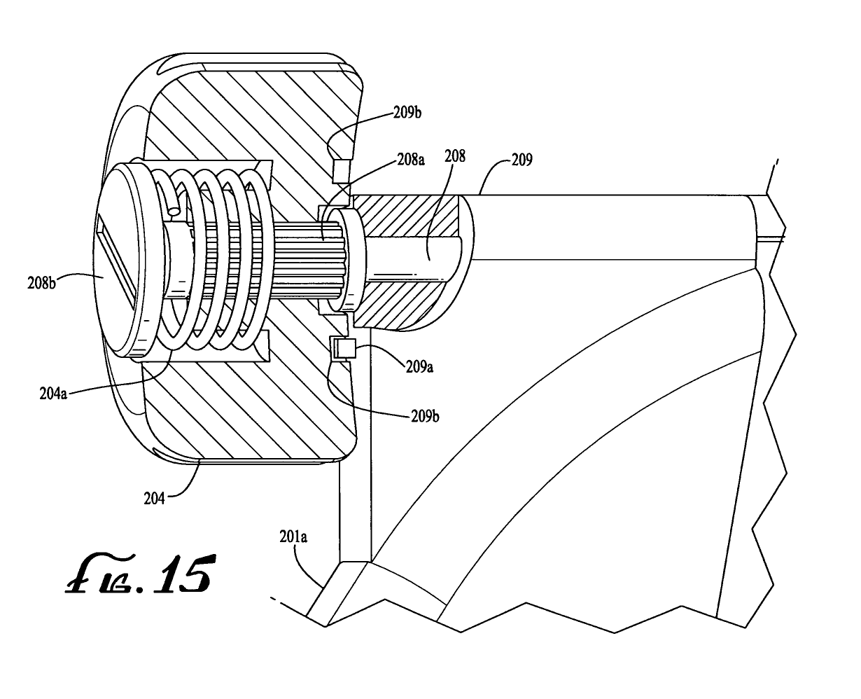

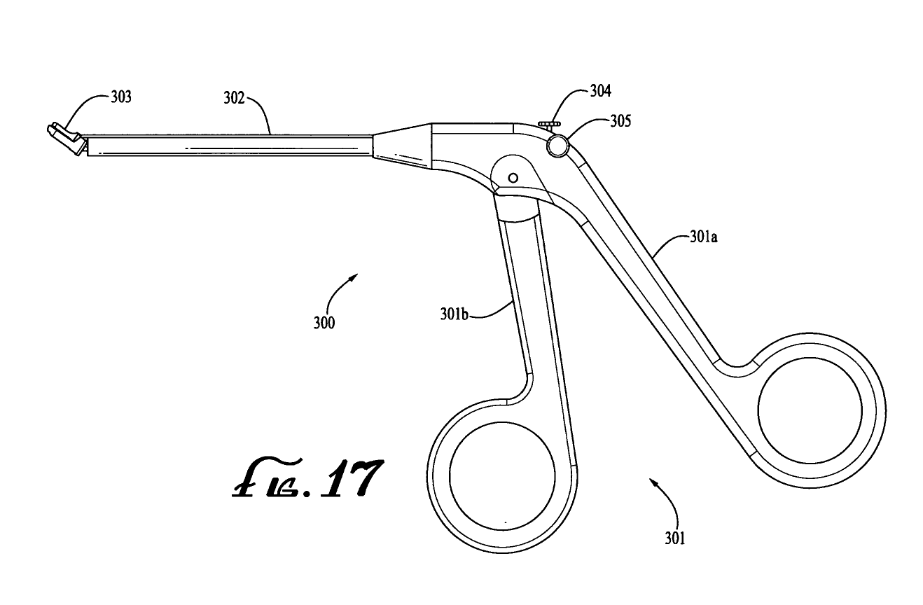

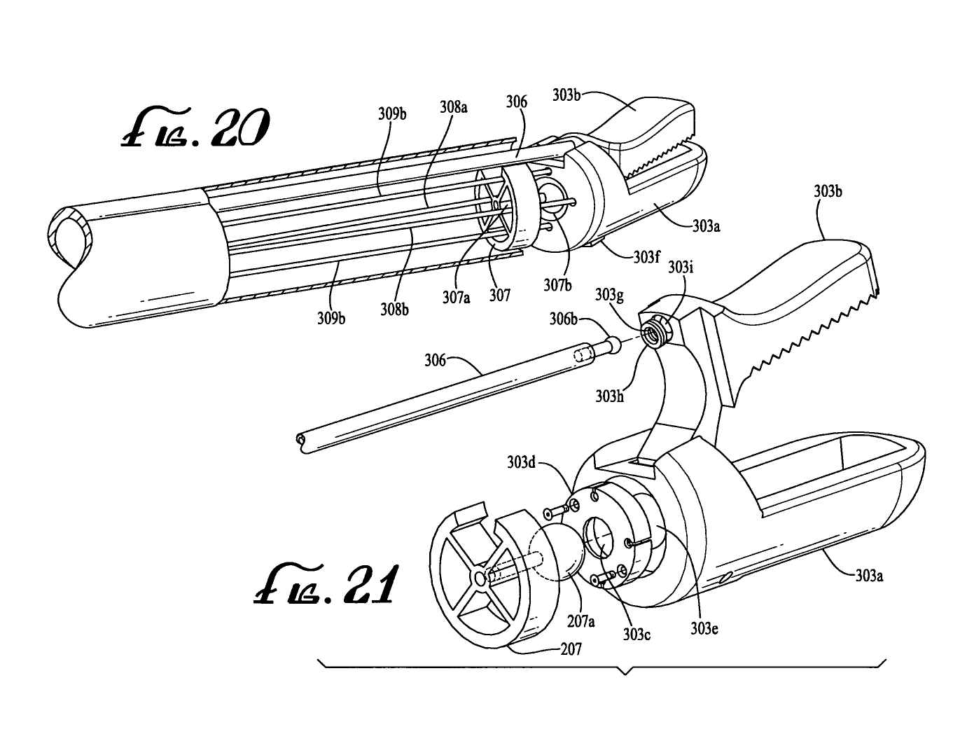

With reference to FIGS. 1-21, preferred embodiments of the adjustable tissue cutter will be described.

### Rigid/Flexible Adjustable Cutter

FIGS. 1-5 depict a preferred embodiment featuring a tissue cutter with a rigid/flexible shaft region that is manipulated directly by the using surgeon (or surgical staff) to obtain a desired angle relative to the main shaft of the instrument. As depicted in FIG. 1, the rigid/flexible embodiment 100 of the adjustable tissue cutter includes three distinct parts: a hand piece 101 preferably featuring a scissor-grip actuator comprising a fixed part 101a and a cutter jaw operator 101b pivotably joined to the fixed part in order to provide a scissor-like action, the hand piece 101 connected to a shaft 102 in turn connected to a distal-end tissue cutter 103. The shaft 102, in turn, comprises a length of rigid tubing 102a at the hand-piece end connected to a length of rigid/flexible tubing 102b at the distal end.

The scissor-grip of the hand piece 101 of the preferred embodiment will be familiar to surgical staff as it is the same type of grip used on arthroscopic scissors, forceps and the like already in common use. In one embodiment the hand piece 101 is primarily composed of plastic. Other materials may be used in other embodiments.

In one embodiment, the three components of the tool (the hand piece 101 and the two regions of the shaft 102) are permanently connected to one another as by welds or other means. In other embodiments, one or more of the connections may be a detachable connection allowing replacement of the individual components.

The rigid/flexible portion 102b of the shaft 102 is intended to be bent by the surgeon (or surgical staff) to a particular desired angle at the time of use. The material of the rigid/flexible portion 102b of the shaft is 102 sufficiently flexible so that it may readily be bent by hand, but sufficiently rigid so that, once in its desired configuration, it will maintain that configuration under the pressures ordinarily exerted on the device during surgery.

In the preferred embodiment, the rigid/flexible portion 102b of the shaft 102 tapers down from a wider region at the end attached to the rigid portion 102a of the shaft 102 to a narrower end where it meets the distal cutting end 103. In other embodiments the rigid/flexible portion 102b of the shaft 102 may be of a constant diameter.

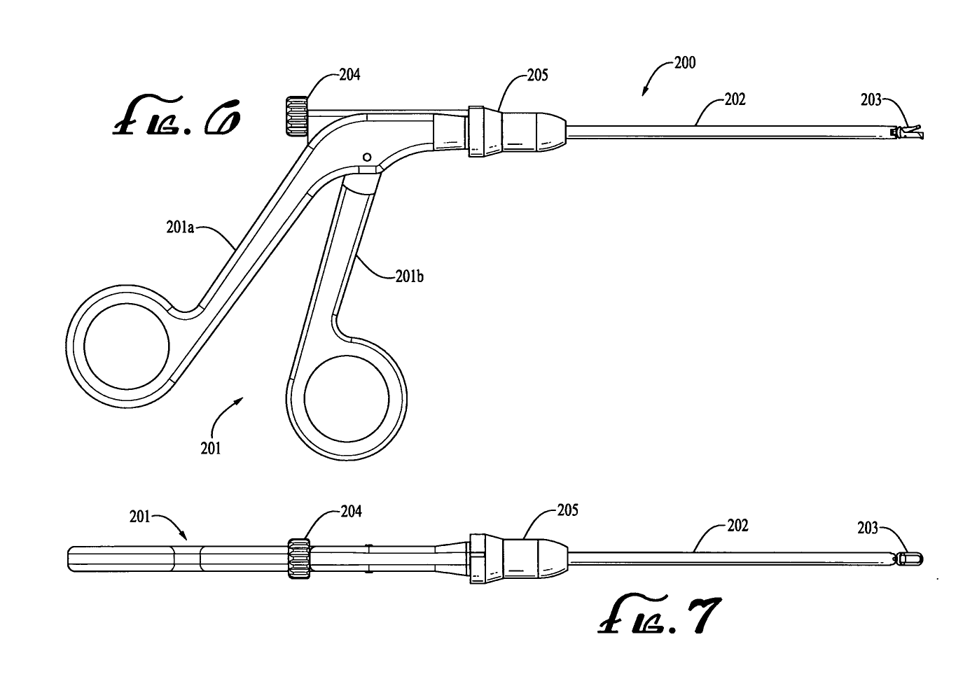

FIG. 2 depicts the device from above, configured with the rigid/flexible portion 102b of the shaft 102 bent to the right. In one embodiment, the rigid/flexible portion 102b of the shaft 102 has a limited range of adjustment of from zero (0) to thirty (30) degrees offset in any direction from the centerline of the rigid portion 102a of the shaft 102. Other embodiments may allow a different range of motion or allow unlimited flexibility. In some embodiments the rigid/flexible shaft will be bendable both horizontally and vertically, while, in alternate embodiments, it may be bendable only in a single plane.

As depicted in FIGS. 3, 4 and 5, both portions of the shaft 102 are hollow, and a length of flexible cable 104 connecting the cutter jaw actuator 101b of the hand piece 101 to the tissue cutter 103 runs through a channel 105 in the center of the shaft 102. The cable 104 functions to translate the opening-closing motion of the hand piece 101 into a cutting action of the distal-end tissue cutter 103.

The channel 105 of the rigid/flexible portion 102b of the shaft is filled with an internal cable-support medium to maintain the position of the cable. In the preferred embodiment this cable support is made of a flexible plastic, though other materials may be used in other embodiments. The rigid portion 102a of the shaft 102 may be constructed either of thin-walled tubing, that may include the same, or a similar, cable-support medium inside the channel, or it may be constructed of thick-walled tubing such that the internal channel of the tubing is narrow enough that no separate cable support is needed.

### Tube-Gear Adjustment Tissue Cutter

FIGS. 6-16 depict an alternate preferred embodiment tissue cutter, in which the horizontal angle of the distal-end tissue cutter relative to the centerline of the device is set by use of a knob at the actuator end which controls gears within the body of the device, providing precise and reproducible adjustment of that angle.

As depicted in FIGS. 6-9, the knob/gear embodiment 200 of the adjustable tissue cutter includes five primary parts: a hand piece 201 preferably featuring a scissor-grip actuator comprising a fixed part 201a and a cutter-jaw operator 201b pivotably joined to the fixed part in such a manner as to provide a scissor-handle-like motion of the two parts; a distal-end cutting tool 203; a shaft 202 connected to the hand-piece 201 at one end and to the cutting tool 203 at the distal-end; a knob assembly 204 preferably positioned at the actuator end of the cutter; and a control-transfer assembly 205 situated at the juncture between the hand piece 201 and the shaft 202. The knob assembly 204 is connected through the control-transfer assembly 205 so as to control the horizontal angle of the distal-end tissue cutter 203 relative to the centerline of the device as shown in FIG. 9 and in a manner discussed below. The mechanism of opening and closing the distal-end tissue cutter is described first.

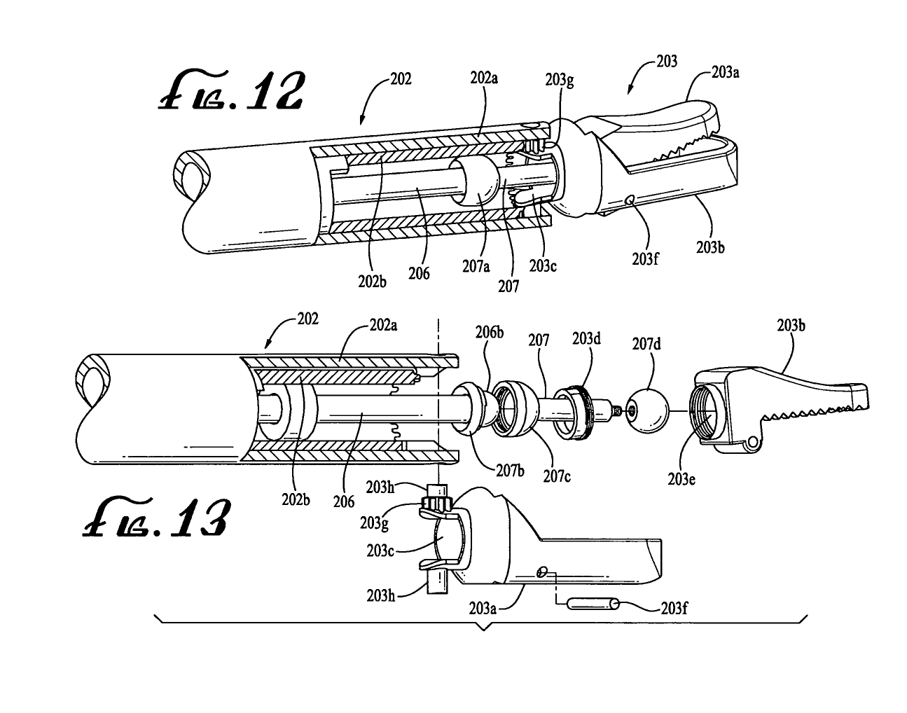

In one embodiment, as depicted in FIGS. 10-13, the cutter-jaw operator 201b is operatively coupled to the distal-end tissue cutter 203 by a pair of rods 206 207 running through the center of the shaft 202. The first, main rod 206 runs the length of the device from the top of the cutter-jaw operator 201b nearly to the point where the distal-end tissue cutter 203 is joined to the shaft 202, and where the main rod 206 connects to the short ball-joint rod 207 which passes through the joint between the shaft 202 and the distal-end tissue cutter 203 and is, in turn, coupled at its distal end to the distal-end tissue cutter 203.

At its rear end, the main rod 206 preferably features a t-shaped head 206a that preferably fits into a slot 201c at the upper end of the cutter-jaw operator 201b—so that pivoting motion of the operator 201b is translated into lengthwise sliding motion of the main rod 206 within the shaft 202. (In other alternate embodiments the t-shaped head 206a and slot 201c may be replaced with other suitable means of pivotably coupling the main rod to the top of the cutter-jaw operator 201b.)

The connection between the main rod 206 and the ball joint rod 207 is preferably made by way of a ball 206b on the end of the main rod that fits into a socket 207a on that end of the ball-joint rod 207. In one embodiment, the socket is comprised of two threaded parts, a collar 207b slipped over the main rod 206 behind the ball 206b and a receiver 207c, which two parts screw together to capture the ball 206b.

At its other end, the ball-joint rod 207 terminates in a second ball 207d that fits into a cavity 203e in the back of the upper jaw 203b of the distal-end tissue cutter 203 so that the ball-joint rod 207 protrudes through an opening 203c in the back of the lower jaw 203a. In one embodiment, similar to the construction of the ball joint, there is a threaded collar 203d with an inner diameter larger than that of the shaft of the ball joint rod 207 but smaller than the outer diameter of the ball 207d. Collar 203d screws onto rear of the upper jaw 203b capturing the ball 207d in the cavity 203e in the upper jaw 203b.

The upper jaw 203b of the distal-end tissue cutter 203 is pivotably joined to the lower jaw 203a by means of a pin 203f, around which the upper jaw 203b pivots.

Through cooperation of the operative components, the basic opening and closing of the distal-end tissue cutter 203 in response to the motion of the scissor-grip actuator 201 is described as follows.

When the scissor-grip actuator 201 is in the initial, closed position, the jaws 203a 203b of the distal-end tissue cutter 203 are, correspondingly closed. As the scissor-grip actuator is opened—by pushing the lower part of the cutter-jaw operator 201b forward—the top of cutter-jaw operator 201b pivots backward. This pulls the t-shaped head 206a of main rod 206—captive in the slot 201c at the top end of the cutter jaw operator 201b—backwards as well. The distal end of the main rod therefore moves backwards correspondingly. The ball 206b at that distal end—captive in the receiver 207a of the ball-joint rod 207—thereby transmits the motion of the main rod 206 to the ball-joint rod 207. Thus the ball joint rod 207, and its distal-end ball 207d, move backwards as well.

The backwards motion of the distal-end ball 207d of the ball-joint rod 207—in turn, captive within the receiver of the upper jaw 203b of the distal-end tissue cutter 203—therefore exerts a backwards force on the rear of the upper jaw 203b, causing it to pivot around the pin 203f connecting it to the lower jaw 203a and, thereby, opening the jaws of the distal-end tissue cutter 203.

In this embodiment, as depicted in FIGS. 10-16, the adjustment of the horizontal angle of the distal-end tissue cutter 203 relative to the centerline of the device is preferably accomplished as follows. Mounted above the actuator 201 at the rear of the device is an angle-adjustment rod 208 housed in a tube 209. At the rear, this rod terminates in a gear 208a which has external teeth and extends through a toothed aperture in the knob 204 so that the external teeth of the gear engage with the internal teeth of the aperture. The gear and aperture engage loosely enough that the knob 204 can slide forward and backward within this aperture 208a. A cavity at the rear of the knob 204 holds a spring 204a which is fitted around the extension 208a and held in place by a cap 208b screwed into the end of the extension 208a.

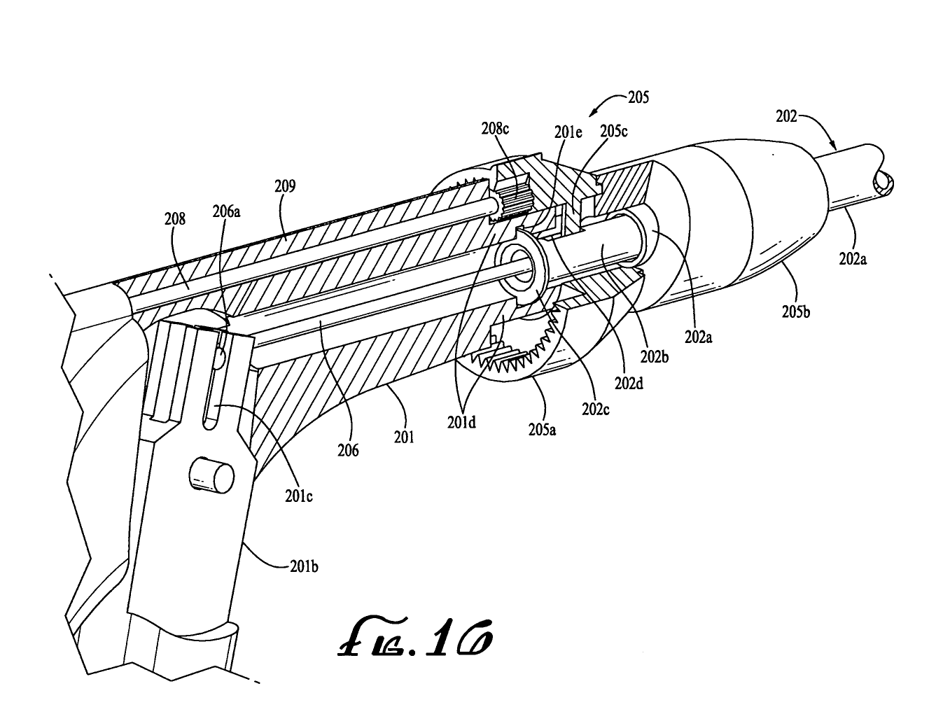

Thus, when there is no external pressure applied to the knob, the spring 204a pushes the knob forward toward the distal end of the tissue cutter. Slightly below the tube 209 on the rear of the body of the device there is a small protrusion/tab stop 209a—in one embodiment in the shape of a cube. On the distal side of the knob 204, a series of multiple depressions 209b—sized and positioned to accept the protrusion/tab stop 209a—are arrayed in a concentric ring around the aperture through which the extension 208a fits. When the knob 204 is pulled back manually (as depicted in FIG. 14), it may, at that point, be freely rotated. When the backward force is released, the knob 204 will slide forward under the pressure of the spring 204a. As the knob 204 is further rotated, the protrusion/tab stop 209a will slip into one of the complementary depressions 209b (as depicted in FIG. 15), barring further rotation of the knob 204, which will be locked in place until manually pulled back again.

The rotation of the knob 204, in turn, causes the attached angle-adjustment rod 208 to rotate inside the tube 209. At its distal end, where it emerges from the tube 209, the angle-adjustment rod 208 terminates in a gear 208c having external teeth. The teeth of the gear 208c mesh with the internally extending teeth on the inside of the ring gear 205a situated at the rear of the control-transfer assembly 205 situated at the juncture between the hand piece 201 and the shaft 202. Rotation of the knob 204 thereby causes rotation of the ring gear 205a in the same direction.

The main shaft 202 of the device is comprised of two tubes 202a 202b concentric around the main rod 206. Outer tube 202a is fixedly joined to the front part 205b of the control-transfer assembly 205 which is, in turn, fixedly joined to the hand piece 201 through element 201d passing through the opening in the ring gear 205a. The inner tube 202b rotates freely around its long axis within the outer tube 202a and around the main rod 206. The outer, fixed tube 202a terminates at its rear where it is fixed to the forward part 205b of the control transfer assembly 205.

The inner, rotating tube 202b terminates further back, with a flange 202c that fits into a groove 201e in the hand assembly member 201c, which flange and groove hold the inner tube 202b in place front-to-back while allowing the inner tube 202b to rotate freely within the outer tube 202a. Tab 205c protrudes from the inside of the forward part of the body of the ring gear 205a into a slot 202d in the inner, rotating tube 202b. The tab 205c transmits rotation of the ring gear 205a to the inner tube 202b.

The distal end of inner tube 202b has gear teeth that engage the gear teeth of gear 203g that is mounted on upper-rear portion of the lower jaw 203a of the distal-end tissue cutter 203. Rotation of the inner tube 202b around its axis is thus transformed by 90° and causes the distal-end tissue cutter 203 to horizontally pivot around the pins 203h by which it is mounted into the outer, fixed tube 202a.

The horizontal angle of the distal-end tissue cutter 203 relative to the centerline of the device imparted by a particular degree of rotation of the knob 204 depends on the gear ratios among the various gears and those, and any other gear ratios of the device, may vary among embodiments of the invention.

### Knob/Wire Adjustment Cutter

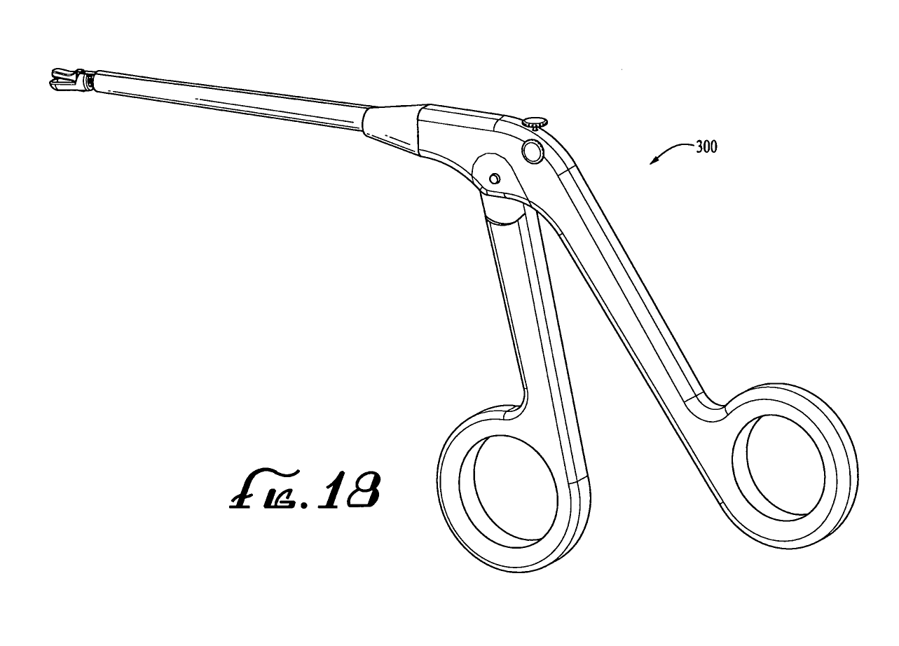

FIGS. 17-21 depict an alternate preferred embodiment in which the horizontal and vertical angle of the distal-end cutter relative to the centerline of the device is set by use of knobs at the actuator end which control wires within the body of the device, providing precise and reproducible adjustment of those angles.

As depicted in FIGS. 17 and 18, the knob/wire embodiment 300 of the adjustable tissue cutter preferably includes five primary parts: a hand piece 301 preferably featuring a scissor-grip actuator comprising a fixed part 301a and a cutter jaw operator 301b pivotably-joined to the fixed part in such a manner as to provide a scissor-handle-like motion of the two parts; a distal-end cutting tool 303; a shaft 302 connected to the hand-piece 301 at one end and to the cutting tool 303 at the distal-end; a horizontal-angle control knob assembly 304 and a vertical-angle control knob assembly 305 both situated on the upper body of the hand piece in line with the shaft 302. The knob assemblies 304, 305 are connected so as to control the horizontal and vertical angles, respectively, of the distal-end tissue cutter 303 relative to the centerline of the device as described below.

In this embodiment, the distal-end tissue cutter preferably is attached to the shaft as follows. An x-shaped attachment mounting 307 is fixedly mounted at the distal end of the shaft. A short, fixed ball joint rod 307a protrudes outward from the attachment mounting 307 and terminates in a ball 307b that fits into the lower jaw 303a of the distal-end tissue cutter 303 so that the rod 307a protrudes through an opening 303c into a cavity 303e in the back of the lower jaw 303a of the distal-end tissue cutter 303. This mounting fixes the distal-end tissue cutter to the device while enabling it to pivot freely both horizontally and vertically relative to the centerline of the device. In alternate embodiments the attachment mounting 307 may not be x-shaped but rather may take on any other shape that functions to provide a base for the ball-joint rod 307a while having sufficient appropriately-positioned openings through which the wires, discussed below, may pass.

In one embodiment, there is a collar 303d with a diameter larger than that of the shaft of the rod 307a but smaller than the diameter of the ball 307b. That collar 303d screws onto the rear of the lower jaw 303a capturing the ball 307b in the cavity 303e in the lower jaw 303a.

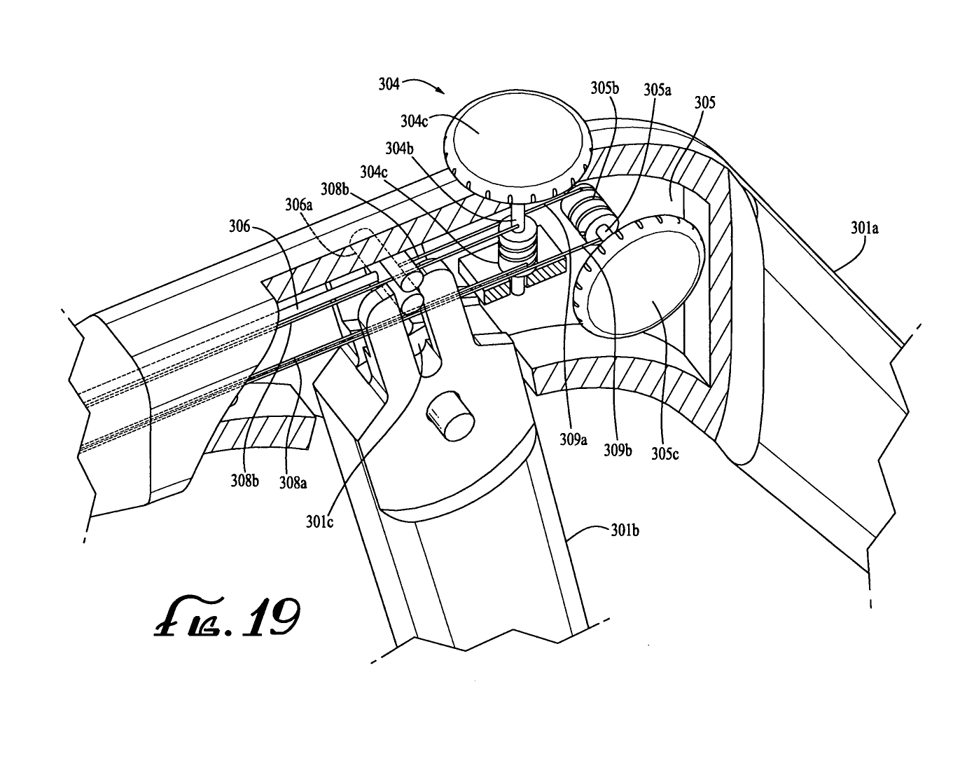

The mechanism of opening and closing the distal-end tissue cutter is as follows. In one embodiment, as depicted in FIGS. 19, 20 and 21 this mechanism operates in a manner similar to that described in the knob/gear embodiment above. In such embodiment, the cutter-jaw operator 301b is operatively coupled to the distal-end tissue cutter 303 by a rod 306 running through the upper portion of the shaft 302. The rod 306 runs the length of the device from the top of the cutter-jaw operator 301b to the rear of the distal-end tissue cutter 303.

At its rear end, the rod 306 preferably features a t-shaped head 306a that fits into a slot 301c at the end of the cutter jaw operator 301b—so that the pivoting motion of the operator 301b is translated into lengthwise reciprocation of the main rod 306 within the hollow shaft 302. (In other alternate embodiments the t-shaped head 306a might be replaced with a ball, or any other means that functions to pivotably couple to the top of the cutter-jaw operator 301b.)

At its other, distal end, the rod 306 passes through the upper opening of the x-shaped attachment mounting 307 and terminates in a ball 306b that fits into the upper jaw 303b of the distal-end tissue cutter 303 so that the rod 306 protrudes through an opening 303g into a cavity 303i in the back of the upper jaw 303b of the distal-end tissue cutter 303. In one embodiment, there is a threaded collar 303h with a diameter larger than that of the shaft of the rod 306 but smaller than the diameter of the ball 306b. That collar 303h screws onto rear of the upper jaw 303b capturing the ball 306b in the cavity 303i in the upper jaw 303b. That upper jaw 303b of the distal-end tissue cutter 303 is pivotably joined to the lower jaw 303a by means of a pin 303f, around which the upper jaw 303b pivots.

Cooperation of the operative structural components of the tissue cutter functions to provide opening and closing of the distal-end tissue cutter 303 in response to the motion of the scissor-grip actuator 301 is as follows. When the scissor-grip actuator 301 is in the initial, closed position, the jaws 303a, 303b of the distal-end tissue cutter 303 are closed.

As the scissor-grip actuator is opened—by pushing the lower part of the cutter-jaw operator 301b forward—the top of that cutter-jaw operator 301b pivots backward. This pulls the t-shaped end 306a of the rod 306—captive in the slot 301c at the top end of the cutter jaw operator 301b—backward as well. The distal end of the rod 306 therefore moves backwards as well. The ball 306b at that end—captive within the receiver of the upper jaw 303b of the distal-end tissue cutter 303—therefore exerts a backward force on the rear of the upper jaw 303b causing it to pivot around the pin 303f connecting it to the lower jaw 303a and, thereby, opening the jaws of the distal-end tissue cutter 303.

In this embodiment, as depicted in FIGS. 17-21, the adjustment of the horizontal angle of the distal-end tissue cutter 303 relative to the centerline of the device preferably is accomplished as follows. A short shaft 304a is mounted vertically into the rear-upper portion of the actuator 301 in such a manner as allows the shaft to rotate around its axis. At the lower end, the shaft 304a terminates in a small spool 304b inside the actuator 301; at its upper end it terminates in knob 304c located above the actuator 301.

A wire 308 is wrapped at least once around the spool 304b such that two segments 308a 308b extend forward from the spool 304b through the shaft 302 at approximately the vertical center of the shaft and to the left and right, respectively, of the horizontal center of the shaft. Viewed from above and to the rear of the device, one wire segment 308a extends forward from the left-hand side of the spool 304b while the other wire segment 308b extends forward from the right-hand side of the spool 304b. At the distal end of the device, the wire segments protrude, respectively, through the left-hand and right-hand openings in the x-shaped attachment mounting 307. The end of each wire segment is, in turn, fixedly attached to the rear of the lower jaw 303b of the distal-end tissue cutter 303 by conventional means.

The adjustment of the horizontal angle of the distal-end tissue cutter 303 operates as follows. As the horizontal-angle control knob 304c is rotated in a clockwise direction, the corresponding attached shaft 304a and spool 304b rotate in the same manner. The surface of the spool 304b and surface of the wire 308 are such that the friction between them is sufficient to prevent the wire from slipping relative to the spool during rotation of the spool, thus causing the wire segments to move during rotation of the spool. As the spool rotates in a clockwise manner, the left-hand (viewed from above, rear) wire segment 308a will unspool from the spool such that the length of the segment will increase, while the right-hand wire segment 308b will be drawn onto the spool and shortened by the same amount.

As this occurs, the attachment of the wire segments 308a, 308b to the left-hand and right-hand sides, respectively, of the rear of the lower jaw 303a of the distal-end-tissue cutter 303 will, thereby, cause the right-hand side of the distal-end tissue cutter 303 to be drawn toward the rear of the device while the corresponding slack created in the left-hand segment of the wire will permit the left-hand side of the distal-end tissue cutter 303 to move an equal distance away from the rear of the device.

The mechanism for the adjustment of the vertical angle of the distal-end tissue cutter 303 preferably operates in an essentially identical manner by means of a horizontally mounted shaft 305a connecting a corresponding spool 305b and knob 305c. A second wire 309 is wrapped around the spool 305b so that upper and lower segments 309a 309b extend from the spool approximately along the horizontal center of the shaft 302 above and below, respectively, the vertical center of the shaft and pass through the upper and lower openings of the x-shaped attachment mounting 307. The end of each wire segment is, in turn, fixedly attached to the rear of the lower jaw 303b of the distal-end tissue cutter 303 by any appropriate means. The rotation of the knob 305c causes motion of the wire segments 309a, 309b and in turn the change in the vertical angle of the distal-end tissue cutter 303 relative to the centerline of the device.

In some embodiments the knob assemblies 304, 305 may include spring-loaded, tabbed, positional-locking structures as previously described with respect to the knob/gear adjustment embodiments.

In some embodiments, each single wire 308, 309 may be replaced by two separate wire segments each independently, fixedly joined to the corresponding spool. In such embodiments the surface of the spool and wire need not be frictionally linked, as one end of each wire segment will be fixed to the spool and friction will not be necessary to transfer force from the rotation of the spool to the wire.

In some embodiments, only one of either the horizontal angle or the vertical angle of the distal-end tissue cutter may be adjustable and there would, in such embodiments, be only a single knob controlling a single spool and wire-segment-pair assembly to adjust that angle.

What is claimed is:

1. An adjustable tissue cutter tool for removal of damaged tissue within a joint comprising:
a permanently-attached distal-end cutting mechanism comprising a sharp top member and a sharp bottom member adapted to cut tissue;
a permanently-attached hand piece including a scissor-type actuator adapted to cause a cutting motion of said distal-end cutting mechanism;
a hollow, longitudinal connection member connecting said hand piece to said distal-end cutting mechanism; and
a translation mechanism, positioned within said hollow, longitudinal connection member;
said translation mechanism adapted to translate squeezing motion of the actuator of said hand piece into cutting motion of said distal-end cutting mechanism;
said tool adapted to allow the horizontal and/or vertical angle of said distal-end cutting mechanism relative to the centerline of said longitudinal connection member to be adjusted to a desired angle and to retain said desired angle during tissue cutting.

2. The adjustable tissue cutter tool of claim 1 wherein said translation mechanism comprises:
a cable routed through said hollow, longitudinal connection member;
said cable connected at its proximal end to the actuator of said hand piece, and connected at its distal end to the distal-end cutting mechanism, said cable adapted to translate squeezing motion of the actuator of said hand piece into cutting motion of said distal-end cutting mechanism; and,
said hollow, longitudinal connection member further comprises an internal cable-support structure adapted to guide and support said cable.

3. The adjustable tissue cutter tool of claim 2 wherein said hollow, longitudinal connection member further comprises:
a rigid region and a rigid-flexible region;
said rigid region attached at its proximal end to said hand piece and at its distal end to the proximal end of said rigid-flexible region;
said rigid-flexible region further attached at its distal end to said distal-end cutting mechanism;
said rigid-flexible region adapted to be bendable to a desired angle and to retain said angle during tissue cutting.

4. The adjustable tissue cutter tool of claim 3 wherein said rigid-flexible region is tapered from a wide end at its proximal end to a relatively narrower end at its distal end.

5. The adjustable tissue cutter tool of claim 4 wherein said tapering is linear.

6. The adjustable tissue cutter tool of claim 4 wherein said tapering is non-linear.

7. The adjustable tissue cutter tool of claim 3 wherein one or more of the attachments positioned between the hand piece and distal-end cutting mechanism are welds.

8. The adjustable tissue cutter tool of claim 3 wherein one or more of the attachments positioned between the hand piece and distal-end cutting mechanism are made with fixative.

9. The adjustable tissue cutter tool of claim 3 wherein one or more of the attachments positioned between the hand piece and distal-end cutting mechanism are made with screws.

10. The adjustable tissue cutter tool of claim 2 manufactured of materials adapted for repeated cycles of use and sterilization.

11. The adjustable tissue cutter tool of claim 2 manufactured of materials adapted for single use.

12. The adjustable tissue cutter tool of claim 1 wherein said translation mechanism comprises:
a first rod and a second rod;
said first rod extending lengthwise within said hollow, longitudinal connection member, connected at its proximal end to the actuator of said hand piece, and connected by a ball joint at its distal end to the proximal end of said second rod;
said second rod further connected at its distal end to the distal-end cutting mechanism;
whereby said first rod and said second rod are adapted to translate squeezing motion of the actuator of the hand piece into cutting motion of said distal-end cutting mechanism; and,
wherein the ball joint connecting said first rod and said second rod is adapted to enable the angle of the second rod and of the distal-end cutting mechanism connected to it to be adjusted relative to the centerline of the longitudinal connection member.

13. The adjustable tissue cutter tool of claim 12 further comprising:
an inner tube positioned concentrically inside said hollow longitudinal connection member and,
a pair of vertical pins attached respectively to the top and bottom of the rear portion of said distal-end cutting mechanism;
said pair of vertical pins adapted to attach said distal-end cutting mechanism to the distal end of said hollow, longitudinal connection member whereby said distal-end cutting mechanism can pivot horizontally on said pins;
a cylindrical lever positioned concentrically around the outside of the hollow, longitudinal member and comprising a tab;
said tab passing through a slot in the hollow, longitudinal connection member and engaging with the inner tube;
whereby said tab is adapted to translate rotation of said lever into rotation of said inner tube;
a gear positioned vertically at the rear of the distal-end cutting tool mechanism;
said inner tube having gear teeth cut into its distal end adapted to mesh with the teeth of said gear; and,
whereby rotation of the inner tube translates into horizontal pivoting of the distal-end cutting mechanism away from the centerline of the longitudinal connection member.

14. The adjustable tissue cutter tool of claim 13 wherein rotation of the concentric cylindrical lever is accomplished manually.

15. The adjustable tissue cutter tool of claim 13 further comprising a control mechanism adapted to accomplish rotation of the concentric cylindrical lever, said control mechanism comprising:
teeth cut into the inner surface of said concentric cylindrical lever;
a channel, passing longitudinally through the upper portion of the hand piece;
a small gear;
a shaft, positioned in said channel and terminating at its distal end in said small gear;
a knob, mounted to the proximal end of said shaft at the upper-rear of the tool behind the hand piece;
said shaft free to rotate along its long axis within said channel; and,
the teeth of said small gear positioned to engage said teeth on the inner surface of said concentric cylindrical lever, whereby rotation of the knob in a given direction causes rotation of said concentric cylindrical lever in the same direction.

16. The adjustable tissue cutter tool of claim 15 wherein said knob is directly attached to said shaft.

17. The adjustable tissue cutter tool of claim 15 further comprising:
a shaped head, attached to the proximal end of said shaft;
an opening in the distal face of said knob;
an array of one or more indents in the distal face of said knob; and,
a small tab protruding from the rear of the hand piece below said shaft;
said opening adapted to closely fit said shaped head and said opening and said shaped head thus adapted to permit said knob to slide along the length of the shaped head, but not to rotate freely about it;
said shaped head passing through said close-fitting opening in said knob; and,
said indents adapted to accept said tab whereby pushing said knob forward and rotating it suitably causes the tab to engage one of said indents.

18. The adjustable tissue cutter tool of claim 17 further comprising:
a cavity within the knob, and a spring, captive in said cavity;
said cavity and spring adapted to resist rearward pressure on the knob and exert forward pressure on it;
whereby, relaxation of rearward pressure on said knob causes the knob to move forward toward the hand piece, and to capture said small tab on the rear of the hand piece in one of said indents when said knob is rotated.

19. The adjustable tissue cutter tool of claim 1 wherein said translation mechanism comprises:
a mounting bracket fixedly attached to the distal end of said longitudinal connection member, a short shaft protruding distally from said mounting bracket, a ball mounted at the end of said short shaft, and said distal-end cutting mechanism pivotably attached to said ball;
a first, vertical shaft, said vertical shaft passing rotatably through and into the upper rear of said hand piece, a first knob attached to the top end of said vertical shaft, and a first spool attached to the lower end of said vertical shaft, within the body of said hand piece;
a second, horizontal shaft passing rotatably through and into one side of the upper rear of the hand piece, a second knob attached to the outer end of said horizontal shaft, and a second spool attached to the inner end of said horizontal shaft, within the body of said hand piece;
a first wire connected to said first spool whereby the two ends of the wire are directed roughly in parallel toward the distal end of the tool;
one or more openings in said mounting bracket adapted to allow the two ends of said first wire to pass through said one or more openings in said mounting bracket and to attach to the left and right regions, respectively, of the rear of the distal-end cutting mechanism;
whereby rotating said first knob clockwise causes said distal-end cutting mechanism to pivot horizontally on said ball relative to the centerline of the hollow, longitudinal connection member;
a second wire connected to said second spool whereby the two ends of the wire are directed roughly in parallel toward the distal end of the tool;
one or more additional openings in said mounting bracket adapted to allow the two ends of said second wire to pass through said one or more additional openings in said mounting bracket and attach to the upper and lower regions, respectively, of the rear of the distal-end cutting mechanism,
whereby, rotating said second knob so that its distal edge rotates upwards causes said distal-end cutting mechanism to pivot vertically on said ball relative to the centerline of the hollow, longitudinal connection member.

20. The adjustable tissue cutter tool of claim 19 wherein:
the surface of each spool is such that, when its respective wire is wrapped one or more times around said spool, there is sufficient friction between the surface of said spool and said wire that said wire will not slip relative to the spool when said spool is rotated, and the connection of each wire to its respective spool is accomplished by such wrapping.

21. The adjustable tissue cutter tool of claim 19 wherein:
the first wire comprises two parts, each of which is attached directly to said first spool; and,
the second wire comprises two parts, each of which is attached directly to said second spool.
